(19) United States
(12) Patent Application Publication
(10) Pub. No.: US 2024/0277901 A1
(43) Pub. Date: Aug. 22, 2024

(54) Engineered Neuronal Microtissue Provides Exogenous Axons for Delayed Nerve Fusion and Rapid Neuromuscular Recovery

(71) Applicants: THE TRUSTEES OF THE UNIVERSITY OF PENNSYLVANIA, Philadelphia, PA (US); The United States Government as represented by the Department of Veterans Affairs, Washington, DC (US); Mary Hitchcock Memorial Hospital, for itself and on behalf of Dartmouth-Hitchcock Clinic, Lebanon, NH (US)

(72) Inventors: Daniel Kacy Cullen, Media, PA (US); Justin C. Burrell, Philadelphia, PA (US); Joseph M. Rosen, Lyme, NH (US)

(21) Appl. No.: 18/568,560

(22) PCT Filed: Jun. 10, 2022
(86) PCT No.: PCT/US22/32978

§ 371 Date: Dec. 8, 2023

Related U.S. Application Data
(60) Provisional application No. 63/209,639, filed on Jun. 11, 2021.

(51) Int. Cl. A61L 27/38; A61L 27/36; A61L 27/52

(52) U.S. Cl. CPC A61L 27/3878; A61L 27/3633; A61L 27/3695; A61L 27/383; A61L 27/52; A61L 2430/32

(57) ABSTRACT

In various aspects and embodiments, the invention provides a tissue engineered neuromuscular interface comprising: an extracellular matrix core; the extracellular matrix core comprising: a population of neurons at a first end of the extracellular matrix core, the population of neurons having axons extending at least a portion of the way along the extracellular matrix core; wherein the population of neurons is selected from the group consisting of one or more motor neurons, one or more motor neurons co-cultured with one or more sensory neurons, and a co-aggregate comprising one or more motor neurons and one or more sensory neurons.

### CROSS-REFERENCE TO RELATED APPLICATION

The present application is entitled to priority under 35 U.S.C. § 119(e) to U.S. Provisional Patent Application No. 63/209,639 filed Jun. 11, 2021, the content of which is incorporated by reference herein in its entirety.

### STATEMENT REGARDING FEDERALLY SPONSORED RESEARCH OR DEVELOPMENT

This invention was made with government support under W81XWH-16-1-0796 awarded by the Army and Merit Award I01-BX003748 provided by the United States Government as represented by the Department of Veterans Affairs. The government has certain rights in the invention.

### BACKGROUND OF THE INVENTION

Peripheral nerve injury (PNI) has been estimated to present in 3% of trauma case and up to 5% if including plexus and root avulsion injuries. More than 550,000 PNI procedures are performed annually in the U.S. Despite recent advancements in neurosurgery, it is estimated that only 50% of patients will achieve satisfactory functional recovery. Although several factors impact successful regeneration, delayed surgical repair is considered the most important contributing factor to poor functional recovery. After injury, axons in the distal nerve undergo Wallerian degeneration. Dedifferentiated Schwann cells temporarily form columnar pro-regenerative structures called the bands of Büngner that promote axon regeneration and targeted muscle reinnervation. Over time, prolonged denervation due to the loss of axonal contact subsequent to the delayed surgical repair leads to the degradation of the bands of Büngner, diminishing the potential for muscle reinnervation and ultimately functional recovery. Unfortunately, delayed surgical repairs are common because surgeons utilize a “wait-and-see” approach to ascertain potential spontaneous recovery. Thus, there is a clinical unmet need for a novel strategy that can prolong the optimal surgical window and improve the likelihood for meaningful recovery. Accordingly, there is a need in the art for improved methods of nerve repair. This disclosure addresses that need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1F: Tissue Engineered Neuromuscular Interfaces (TE-NMIs) Fabrication and Characterization. FIG. 1A: TE-NMIs are anatomically-inspired bioengineered pathways with discrete neuron populations spanned by sensory, motor, or both motor and sensory axon tracts within a protective biomaterial encasement. The modular TE-NMI fabrication process allows for construction of micro-column hydrogels with various diameters, neuronal cell sources, or biomaterial outer encasement. FIG. 1B: Representative phase and (B′) confocal images are shown of a sensory TE-NMI with a 2 mm outer diameter and 1 mm inner diameter labeled with Tuj1, a neuronal/axonal marker (green) and counterstained with hoechst (blue) to identify nuclei. FIG. 1C: Representative confocal image of a motor TE-NMI with a 350 μm outer and 180 μm inner diameter at 7 days in vitro (DIV) that was virally transduced to express green fluorescent protein (GFP). FIG. 1D: At 14 days in vitro, phase imaging revealed two discrete populations of motor neurons (MNs) spanned by axons. (D′) High resolution confocal imaging revealed discrete regions of motor neurons/axons labeled for Tuj1, ChAT, a motor neuron specific marker, and hoechst. FIG. 1E: Representative confocal images at 7 DIV of constructs with an agarose or an agarose-gelatin composite (AGX) outer encasement. FIG. 1F: Neurite length within each biomaterial encasement was compared at 1 and 3 DIV. Mean values compared using two-way ANOVA. Error bars represent standard error. *p<0.05; **p<0.01; ****p<0.0001. Scale bars: FIGS. 1C-1D 500 μm, zoom in: 100 μm. FIG. 1E 500 μm.

FIGS. 2A-2G: TE-NMI Survival, Outgrowth, and Integration with the Otherwise Denervated Nerve. FIG. 2A: Schematic illustrating the chronic host axotomy surgical model and experimental groups, including transplantation of an acellular column, one TE-NMI, or two TE-NMI. Acellular controls were also transplanted as negative controls. We hypothesized that TE-NMI would extend axons that interact with the Schwann cells in the otherwise denervated distal nerve. FIG. 2B: Intraoperative photos showing TE-NMIs can be micro-injected in the nerve. FIG. 2C: Representative image of a micro-injected TE-NMI at 2 weeks post transplantation that was visualized following optical clearing and multiphoton microscopy. Robust TE-NMI neurons and axons (GFP) were found within the lumen protected from host cells entering the graft zone. FIG. 2D: To assess whether TE-NMI axons extended in the otherwise denervated nerve and interacted with the Schwann cells, nerve cross-sections taken 5 mm distal to the transplant site were labeled for Schwann cells (S100) and TE-NMI axons (GFP). FIG. 2E: High resolution image showing an example of GFP+TE-NMI axons extending through aligned Schwann cells resembling the bands of Büngner. FIG. 2F: Greater GFP outgrowth was found distal to two TE-NMIs than one TE-NMI. FIG. 2G: Increased S100 coverage distal to the transplant site was found in the two TE-NMI group. These findings suggest TE-NMIs can interact with and potentially influence the host S100+ Schwann cells in the denervated distal nerve at 6 weeks post transplantation in a model of host nerve axotomy. Error bars represent standard error. Mean values compared using a one-way ANOVA followed by Tukey's post-hoc test. *p<0.05. Scale bars: FIG. 2C: 100 μm, FIG. 2D: 100 μm, FIG. 2E: 5 μm.

FIGS. 3A-3D: Evoked Muscle Response at 16 Weeks Following TE-NMI Transplantation in Chronic Host Nerve Axotomy Model. FIG. 3A: Schematic illustrating the surgical model, transplantation paradigm, and outcome measure. Mixed motor-sensory TE-NMIs were secured to the common peroneal nerve in a model of host chronic nerve axotomy. At 16 weeks post transplantation, the evoked muscle response was recorded following transcutaneous stimulation over the common peroneal nerve innervating the distal target tibialis anterior muscle. FIG. 3B: Representative confocal image of a mixed motor-sensory TE-NMI containing neuron populations transduced to express TD-tomato (motor, red) or GFP (sensory, green). FIG. 3C: Compared to the irregular/lack of recordable waveform in the no implant or micro-column only control groups, a reproducible robust waveform was elicited in the TE-NMI group. FIG. 3D: Greater mean amplitude of the evoked muscle response was found in the TE-NMI group compared to the controls. These findings suggest that TE-NMIs functionally integrate with the denervated muscle and preserve the electrophysiological muscle response at 16 weeks post chronic nerve axotomy. Error bars represent standard error. Mean values compared using a one-way ANOVA followed by Tukey's post-hoc test. **p<0.01.

FIGS. 4A-4I: Delayed Axon Fusion via Freshly-Cut TE-NMIs Axons in the Otherwise Denervated Distal Nerve. FIG. 4A: Schematic illustrating the surgical model, delayed nerve fusion paradigm, and outcome measure. At 20 weeks post transplantation and host chronic nerve axotomy, the TE-NMI was removed leaving behind freshly transected axons in the distal nerve. To enable axon fusion, the graft was excised in hypotonic saline containing a calcium chelating agent, similar to previous protocols. FIG. 4B: Intraoperative image at 20 weeks post transplantation showing the proximal common peroneal nerve secured to a nearby muscle, the TE-NMI secured to the distal nerve, and the uninjured tibial nerve coursing above it. FIG. 4C: Intraoperative image immediately after delayed nerve repair showing the previously uninjured tibial nerve sutured to the distal portion of the common peroneal nerve following TE-NMI excision. The blue staining is from methylene blue application during the fusion protocol. FIG. 4D: Compound nerve action potentials recorded immediately after delayed nerve fusion were obtained in all animals that had received a TE-NMI. Greater nerve conductivity was found in the TE-NMI group compared to acellular controls. FIG. 4E: Compound muscle action potentials were recoded after eliciting an evoked muscle response by stimulating proximal to the repair site. Greater evoked muscle response was observed in the TE-NMI group compared to acellular controls. FIG. 4F: At 20 weeks post repair, the surgical site was re-exposed and the TE-NMI transplant was harvested for histological analyses. Representative longitudinal images are shown labeling neurons and dendrites with MAP2 (far red) and sensory and motor TE-NMI neurons and axons with endogenous expression of GFP and tdTomato, respectively. Robust TE-NMI neuron survival with axons spanning the lumen were found at 20 weeks post transplantation. FIG. 4G: At high magnification, healthy neurons were readily visualized within the micro-column co-labeling with MAP2. FIG. 4H: Representative longitudinal nerve sections and FIG. 4I: axial nerve cross-sections immediately distal to the excised transplant are shown labeled for Schwann cells (S100). Robust TE-NMI sensory outgrowth (GFP, green) was visualized. TE-NMI outgrowth was found (TD-Tomato, red), but the expression was weaker. Error bars represent standard error. Mean values compared using two-tailed unpaired Student's t-tests. *p<0.05; **p<0.01. Scale bars: FIG. 4F 25 μm.

FIGS. 5A-5C: Electrophysiological Functional Recovery at 1 Month Following Delayed Nerve Repair. FIG. 5A: Schematic illustrating the electrophysiological outcome measures obtained at 1 month following delayed nerve repair (24 weeks following initial nerve transection). FIG. 5B: Compound nerve action potentials (CNAPs) were elicited in both groups, however, a greater response and faster conduction velocity was observed in animals that had previously received a TE-NMI transplant. FIG. 5C: Compound muscle action potentials (CMAPs) were recorded in all animals with an elevated evoked response in the TE-NMI group. Mean values compared using two-tailed unpaired Student's t-tests. Error bars represent standard error. *p<0.05; **p<0.01.

FIGS. 6A-6I: Nerve Morphometry and Muscle Reinnervation at 1 Month Following Delayed Nerve Repair. FIG. 6A: Representative confocal images of nerve cross-sections 5 mm distal to the repair site were labeled for Schwann cells (S100), host/fused axons (SMI35), and myelin (myelin basic protein; MBP). FIG. 6B: No differences in the number in the total number of axons were found distal to the repair site. FIG. 6C: An increased host axon size was observed in animals that had previously received a TE-NMI transplantation. FIG. 6D: By plotting the axon diameter frequency distribution, a slight rightward shift can be seen in the TE-NMI group. FIGS. 6E and 6F: Representative confocal images of the tibialis anterior (TA) muscle cross-section stained for acetylcholine receptors (bungarotoxin) to identify the neuromuscular junctions (NMJs) and synaptophysin, a presynaptic marker. FIG. 6G: No significant difference in the total number of AchR counts between groups. FIG. 6H: Greater muscle reinnervation, as indicated by the percent of mature NMJ co-labeled for AchR and synaptophysin, was found in animals that previously received a TE-NMI transplantation. FIG. 6I: Increased muscle weight was found in the TE-NMI group. Collectively, these findings suggest that both while both groups are exhibiting ongoing regeneration, TE-NMIs may enable earlier axon maturation and muscle reinnervation following delayed nerve repair. Mean values compared using two-tailed unpaired Student's t-tests. Error bars represent standard error. *p<0.05; **p<0.01.

FIGS. 7A-7C: Mixed Modality TE-NMI Neurite Growth Comparison. FIG. 7A: Representative confocal reconstruction at 3 DIV of a mixed motor-sensory TE-NMI comprised of a population of motor neurons and sensory neurons plated on each end. Motor neurons and sensory neurons (DRG explant) were transduced to endogenously express GFP (green) or tdTomato (red), respectively. FIG. 7B: Neurite growth rates for motor axons extending to the DRG explant (MN-DRG) and sensory axons extending to the motor neurons (DRG-MN) were calculated. FIG. 7C: A representative confocal image at 14 DIV is shown following immunocytochemistry to visualize motor neurons/axons (ChAT, far red). Error bars represent standard error. *p<0.05. Scale bars: 500 μm.

FIGS. 8A-8J: TE-NMI Survival, Outgrowth, and Integration with the Otherwise Denervated Nerve. FIG. 8A: Schematic illustrating the chronic host axotomy surgical model and experimental groups, including transplantation of an acellular column, one TE-NMI, or two TE-NMI. Acellular controls were transplanted as negative controls. We hypothesized that TE-NMI would extend axons that interact with the Schwann cells in the otherwise denervated distal nerve. FIG. 8B: Representative image of a micro-injected TE-NMI at 2 weeks post transplantation that was visualized following optical clearing and multiphoton microscopy. Robust TE-NMI neurons and axons (GFP) were found within the lumen protected from host cells entering the graft zone. FIGS. 8C-8J: To assess whether TE-NMI axons extended in the otherwise denervated nerve and interacted with the Schwann cells, nerve cross-sections taken 5 mm distal to the transplant site were labeled for TE-NMI axons (GFP), Schwann cells (S100β), nuclei (Hoechst; HST), and C-Jun (a gene encoding for a pro-regenerative transcription factor that is transiently found in denervated Schwann cells). FIG. 8C: High resolution image showing an example of GFP+TE-NMI axons extending through aligned Schwann cells resembling the bands of Büngner. FIGS. 8D-8E: Greater GFP outgrowth per nerve was found distal to two TE-NMIs than one TE-NMI. FIG. 8F: At higher magnification, Schwann cells were readily observed with a subpopulation expressing C-Jun. FIG. 8G: Greater number of cells was found in the 2×TE-NMI cohort compared to the acellular group. FIG. 8H: Elevated C-Jun expression was also observed distal to two TE-NMIs. FIG. 8I: Greater number of Schwann cells (identified by HST+S100β co-localization) was also found in the 2×TE-NMI group. FIG. 8J: A significant number of Schwann cells also co-localized with C-Jun distal to two TE-NMIs. These findings suggest TE-NMIs can interact with and potentially influence the host S100+ Schwann cells in the denervated distal nerve at 6 weeks post transplantation in a model of host nerve axotomy, as well as potentially preserve the pro-regenerative phenotype as evidenced by the C-Jun localization. Error bars represent standard error. Mean values compared using a one-way ANOVA followed by Tukey's post-hoc test. *p<0.05. Scale bars: FIG. 8C: 100 μm, FIG. 8D: 100 μm, FIG. 8E: 5 μm.

FIGS. 9A-9H: Nerve Morphometry and Muscle Reinnervation at 1 Month Following Delayed Nerve Repair. FIG. 9A: Representative confocal images of nerve cross-sections 5 mm distal to the repair site were labeled for Schwann cells (S100), host axons (SMI35), and myelin (myelin basic protein; MBP). FIG. 9B: No differences in SMI35 expression were detected distal to the repair site, suggesting a comparable number of host axons regenerated into the distal sheath. FIG. 9C: The mean area of SMI35+ regions found distal to the repair was greater in the TE-NMI cohort, indicating the host axons in the distal nerve were larger than the controls. FIG. 9D: Greater number of myelinated axons were found distal to the repair in the TE-NMI cohort. FIG. 9E: Increased S100β expression, a common marker of Schwann cells, was observed in the TE-NMI group. FIG. 9F: Representative confocal images of the tibialis anterior (TA) muscle cross-section stained for acetylcholine receptors (bungarotoxin) to identify the neuromuscular junctions (NMJs) and synaptophysin, a presynaptic marker (gray-scaled). Sections were counterstained with phalloidin to visualize muscle fibers. FIG. 9G: No significant difference in the total number of AchR counts between groups. FIG. 9H: Greater muscle reinnervation, as indicated by the percent of mature NMJ co-labeled for AchR and synaptophysin, was found in animals that previously received a TE-NMI transplantation. Collectively, these findings suggest that both while both groups are exhibiting ongoing regeneration, TE-NMIs may enable earlier axon maturation and muscle reinnervation following delayed nerve repair. Fractional area was calculated by measuring the percent area of positive fluorescent expression per ROI averaged over three ROIs. Mean values compared using two-tailed unpaired Student's t-tests. Error bars represent standard error. *p<0.05; **p<0.01.

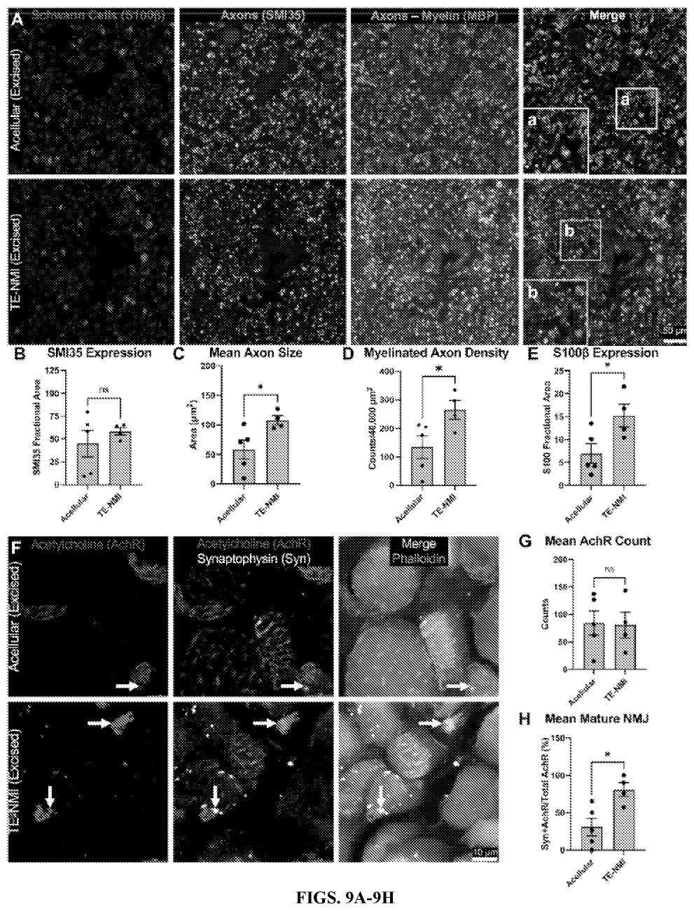

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting. As used herein, the singular form “a,” “an,” and “the” include plural references unless the context clearly dictates otherwise.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

“Isolating,” means to obtain one or more types of cells, purify to remove or substantially remove other cells types and grow in primary culture.

A “subject” or “patient,” as used therein, may be a human or non-human mammal. Non-human mammals include, for example, livestock and pets, such as ovine, bovine, porcine, canine, feline and murine mammals. Preferably, the subject is human.

“Aggregate” and “neuron aggregate” are used interchangeably to refer to an aggregate or sphere of neurons and/or glial cells formed by centrifugation.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Tissue Engineered Neuromuscular Interface

In one aspect, the invention provides a tissue engineered neuromuscular interface comprising: an extracellular matrix core; the extracellular matrix core comprising: a population of neurons at a first end of the extracellular matrix core, the population of neurons having axons extending at least a portion of the along the extracellular matrix core; wherein the population of neurons is selected from the group consisting of one or more motor neurons, one or more motor neurons co-cultured with one or more sensory neurons, and a co-aggregate comprising one or more motor neurons and one or more sensory neurons. In various embodiments, the TE-NMI further comprises a hydrogel sheath coaxially surrounding the extracellular matrix core.

In various embodiments, the tissue engineered neuromuscular interface further comprises a second population of neurons at a second end of the extracellular matrix core, the second population of neurons having axons extending at least a portion of the way along the extracellular matrix core; the second population of neurons selected from the group consisting of one or more motor neurons, one or more motor neurons co-cultured with one or more sensory neurons, and a co-aggregate comprising one or more motor neurons and one or more sensory neurons.

In various embodiments, the population of neurons may be one or more neurons. In various embodiments, the population of neurons may be a neuron aggregate. Neuron aggregates are described in U.S. Publication No. 2019/0126043, which is hereby incorporated by reference. Various methods for producing neuron aggregates are known in the art. By way of nonlimiting example, neuron aggregates may be formed by centrifuging neurons in inverted pyramidal wells. In various embodiments, the neuron aggregates may be co-aggregates comprising more than one type of neuron. Co-aggregates may be formed by dissociating each type of neuron to be included in the co-aggregate and combining the dissociated neurons before forming an aggregate from the mixed population of neurons.

In various embodiments, the co-aggregate has a cross-sectional dimension between about 50 μm and about 100 μm, between about 100 μm and about 150 μm, between about 150 μm and about 200 μm, between about 200 μm and about 250 μm, between about 250 μm and about 300 μm, between about 300 μm and about 350 μm, between about 350 μm and about 400 μm, between about 400 μm and about 450 μm, between about 450 μm and about 500 μm, between about 500 μm and about 700 μm, between about 700 μm and about 1000 μm, between about 1000 μm and about 1500 μm, between about 1500 μm and about 2000 μm, and between about 2500 μm and about 3000 μm.

In various embodiments, the extracellular matrix core has a largest cross-sectional dimension selected from the group consisting of: between about 10 μm and about 25 μm, between about 25 μm and about 50 μm, between about 50 μm and about 100 μm, between about 100 μm and about 150 μm, between about 150 μm and about 200 μm, between about 200 μm and about 250 μm, between about 250 μm and about 300 μm, between about 300 μm and about 400 μm, between about 400 μm and about 500 μm, between about 500 μm and about 700 μm, and between about 700 μm and about 1000 μm, between about 1000 μm and about 1500 μm, and between about 1500 μm and about 2000 μm, and between about 2000 μm and about 2500 μm, and between about 2500 μm and about 3000 μm.

In various embodiments, the hydrogel sheath has a largest cross-sectional dimension selected from the group consisting of: between about 20 μm and about 50 μm, between about 50 μm and about 100 μm, between about 100 μm and about 200 μm, between about 200 μm and about 250 μm, between about 250 μm and about 300 μm, between about 300 μm and about 350 μm, between about 350 μm and about 400 μm, between about 400 μm and about 450 μm, between about 450 μm and about 500 μm, between about 500 μm and about 600 μm, between about 600 μm and about 800 μm, between about 800 μm and about 1200 μm, between about 1200 μm and about 1700 μm, and between about 1700 μm and about 2200 μm, and between about 2200 μm and about 2700 μm, and between about 2700 μm and about 3200 μm. In various embodiments, the hydrogel sheath has a largest cross-sectional dimension of about 701 μm and the extracellular matrix core has a largest cross-sectional dimension of about 300 μm.

In various embodiments, the tissue engineered neuromuscular interface has a length between about 100 μm and about 200 μm, between about 200 μm and about 250 μm, between about 250 μm and about 300 μm, between about 300 μm and about 350 μm, between about 350 μm and about 400 μm, between about 400 μm and about 450 μm, between about 450 μm and about 500 μm, between about 500 μm and about 600 μm, between about 600 μm and about 800 μm, between about 800 μm and about 1200 μm, between about 1200 μm and about 1500 μm, and between about 1500 μm and about 2000 μm.

In various embodiments, the tissue engineered neuromuscular interface further comprises one or more non-neuronal cells selected from the group consisting of: endothelial cells, myocytes, myoblasts, astrocytes, olfactory ensheathing cells, oligodendrocytes, or Schwann cells.

In various embodiments, the neurons are derived from stem cells or are isolated from dorsal root ganglia. In various embodiments, the neurons are xenogeneic neurons, autologous/patient-specific neurons, allogenic neurons, whole dorsal root ganglia or sensory explants. In various embodiments, the neurons are xenogeneic neurons derived from wild type or transgenic pigs.

In various embodiments, the extracellular matrix core comprises collagen, gelatin, laminin, fibrin, fibronectin and/or hyaluronic acid. In various embodiments, the hydrogel sheath comprises agarose, collagen, gelatin, silk, chitosan, fibrin, and/or hyaluronic acid.

### Methods of Preserving Regenerative Capacity of a Distal Nerve Segment by Implanting a Tissue Engineered Neuromuscular Interface

In another aspect, the invention provides a method of preserving the regenerative capacity of a distal nerve segment subsequent to a peripheral nerve injury in a subject in need thereof, the method comprising implanting one or more tissue engineered neuromuscular interface (TE-NMI) into a distal site in the distal nerve segment; wherein the TE-NMI comprises: an extracellular matrix core; the extracellular matrix core comprising: a population of neurons at a first end of the extracellular matrix core, the population of neurons having avons extending at least a portion of the way along the extracellular matrix core; wherein the population of neurons is selected from the group consisting of one or more motor neurons, one or more motor neurons co-cultured with one or more sensory neurons, and a co-aggregate comprising one or more motor neurons and one or more sensory neurons. In various embodiments, the TE-NMI further comprises a hydrogel sheath coaxially surrounding the extracellular matrix core.

In various embodiments, the TE-NMI further comprises: a second population of neurons at a second end of the extracellular matrix core, the second population of neurons having axons extending at least a portion of the way along the extracellular matrix core; the second population of neurons selected from the group consisting of one or more motor neurons, one or more motor neurons co-cultured with one or more sensory neurons, and a co-aggregate comprising one or more motor neurons and one or more sensory neurons.

Without meaning to be limited by theory, in various aspects and embodiments and as further discussed in the Examples herein, implantation of one or more TE-NMIs into the distal segment of an injured peripheral nerve allows the neurons within the TE-NMI to grow axons into the distal nerve segment. These axons preserve the regenerative capacity of the distal nerve segment that may otherwise be lost.

In various embodiments, the implantation is performed immediately after the injury. In various embodiments, the injury results from surgery. In various embodiments, the implantation is performed less than 24 hours after the injury. In various embodiments, the implantation is performed less than 7 days after the injury. In various embodiments, the implantation is performed less than 2 weeks after the injury. In various embodiments, the implantation is performed less than one month after the injury. In various embodiments, the implantation is performed one month or more after the injury.

In various embodiments, the one or more TE-NMIs are implanted into the distal nerve segment end-to-side, are implanted intrafascicularly or are implanted in-continuity. In various embodiments, implantation of the one or more TE-NMIs is ultrasound- or MRI-guided. In various embodiments, at least two tissue engineered neuromuscular interfaces are implanted into the distal nerve segment. In various embodiments, at least five tissue engineered neuromuscular interfaces are implanted into the distal nerve segment. In various embodiments, at least ten tissue engineered neuromuscular interfaces are implanted into the distal nerve segment.

In various embodiments, the method further comprises performing a primary nerve repair procedure to treat the peripheral nerve injury. In various embodiments, the primary nerve repair procedure comprises direct anastomosis, autograft, allograft, nerve conduit, nerve transfer, or a tissue engineered nerve graft.

### Methods of Treating Peripheral Nerve Injury by Implanting a Tissue Engineered Neuromuscular Interface

In another aspect, the invention provides a method of treating a peripheral nerve injury in a subject in need thereof, the method comprising: implanting one or more tissue engineered neuromuscular interface (TE-NMI) into a distal site in the distal nerve segment; wherein the TE-NMI comprises: an extracellular matrix core; the extracellular matrix core comprising: a population of neurons at a first end of the extracellular matrix core, the population of neurons having axons extending at least a portion of the way along the extracellular matrix core; wherein the population of neurons is selected from the group consisting of one or more motor neurons, one or more motor neurons co-cultured with one or more sensory neurons, and a co-aggregate comprising one or more motor neurons and one or more sensory neurons; monitoring exogenous axonal growth throughout the otherwise denervated distal segment for innervation of muscle and/or sensory end organ; removing the one or more tissue engineered neuromuscular interface in the distal nerve segment; and performing a primary nerve repair procedure, thereby treating the peripheral nerve injury. In various embodiments, the TE-NMI further comprises a hydrogel sheath coaxially surrounding the extracellular matrix core.

In various embodiments, the TE-NMI further comprises: a second population of neurons at a second end of the extracellular matrix core, the second population of neurons having axons extending at least a portion of the way along the extracellular matrix core; the second population of neurons selected from the group consisting of one or more motor neurons, one or more motor neurons co-cultured with one or more sensory neurons, and a co-aggregate comprising one or more motor neurons and one or more sensory neurons.

Without meaning to be limited by theory, implantation of the TE-NMI contributes to improved efficacy of the primary nerve repair procedure by preserving the pro-regenerative capacity of the distal nerve segment. Exogenous axons promote the expression of Schwann cells in the distal nerve and integrate with the otherwise denervated muscle and/or sensory end target, increasing the ceiling for functional recovery after delayed nerve repair.

In various embodiments, the primary nerve procedure comprises direct anastomosis, autograft, allograft, nerve conduit, nerve transfer, or implantation of tissue engineered nerve graft. In various embodiments, the TE-NMI is removed less than one week after implantation. In various embodiments, the TE-NMI is removed less than one month after implantation. In various embodiments, the TE-NMI is removed less than one year after implantation. In various embodiments, the TE-NMI is removed one year or more after implantation.

### Methods of Treating Peripheral Nerve Injury by Implanting a Tissue Engineered Neuromuscular Interface and Fusing With a Proximal Nerve

In another aspect, the invention provides a method of treating a peripheral nerve injury in a subject in need thereof, the method comprising: implanting one or more tissue engineered neuromuscular interface (TE-NMI) into a distal site in the distal nerve segment; wherein the TE-NMI comprises: an extracellular matrix core; the extracellular matrix core comprising: a population of neurons at a first end of the extracellular matrix core, the population of neurons having axons extending at least a portion of the way along the extracellular matrix core; wherein the population of neurons is selected from the group consisting of one or more motor neurons, one or more motor neurons co-cultured with one or more sensory neurons, and a co-aggregate comprising one or more motor neurons and one or more sensory neurons; monitoring exogenous axonal growth throughout the otherwise denervated distal segment for innervation of muscle and/or sensory end organ; removing the one or more tissue engineered neuromuscular interface in the distal nerve segment; and fusing the TE-NMI axons in the distal nerve segment with at least one proximal axon. In various embodiments, the TE-NMI further comprises a hydrogel sheath coaxially surrounding the extracellular matrix core.

In various embodiments, the TE-NMI further comprises: a second population of neurons at a second end of the extracellular matrix core, the second population of neurons having axons extending at least a portion of the way along the extracellular matrix core; the second population of neurons selected from the group consisting of one or more motor neurons, one or more motor neurons co-cultured with one or more sensory neurons, and a co-aggregate comprising one or more motor neurons and one or more sensory neurons.

In various embodiments, a primary nerve repair is performed. In various embodiments, the primary nerve procedure comprises direct anastomosis, autograft, allograft, nerve conduit, nerve transfer, or implantation of a tissue engineered nerve graft. In various embodiments, a free radical scavenger is applied prior to the primary nerve repair. In various embodiments, the free radical scavenger is methylene blue.

In various embodiments, the axons that extend from the TE-NMI are transected when the TE-NMI is removed and fused with a proximal nerve segment. Nerve fusion using a stretch-grown tissue engineered nerve graft is described in U.S. Publication No. 2020/0230293, hereby incorporated by reference. Various methods for fusing neurons are known in the art and a skilled person will select an appropriate method. In various embodiments, a reagent is applied before removing the TE-NMI to prevent axonal degeneration. In various embodiments, the reagent comprises hypotonic saline or a calcium chelating agent. In various embodiments, the reagent is hypotonic saline with a calcium chelating agent. In various embodiments, the exogenous neurons are genetically modified to prevent Wallerian degeneration, such as SARM1 knockdown.

In various embodiments, a fusogen is applied during the primary nerve repair to promote membrane sealing. In various embodiments, the fusogen is polyethelene glycol or chitosan. In various embodiments, fusogen application promotes nerve regeneration and functional recovery.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Materials and Methods

The materials and methods used to perform the following examples are here described.

### Embryonic Neuron Isolation and Spinal Motor Aggregation

Spinal cords and dorsal root ganglia (DRG) were isolated from embryonic day 16 Sprague-Dawley rats (Charles River, Wilmington, MA) as previously described. DRG explants were e stored overnight in Hibernate-E and were transduced with AAV2/1.hSynapsin.EGFP.WPRE.bGH (UPenn Vector Core). Motor neuron aggregates were formed following force-aggregation of spinal motor neurons isolated from dissociated spinal cords using an Optiprep density gradient as previously described. Motor neuron aggregates were incubated overnight in media and were transduced with an AAV.hSynapsin.tdTomato vector. Neurons were plated in spinal astrocyte-conditioned Neurobasal media+10% FBS supplemented with 37 ng/mL hydrocortisone, 2.2 μg/mL isobutylmethylxanthine, 10 ng/mL BDNF, 10 ng/mL CNTF, 10 ng/mL CT-1, 10 ng/mL GDNF, 2% B-27, 20 ng/mL NGF, 20 μM mitotic inhibitors, 2 mM L-glutamine, 417 ng/mL forskolin, 1 mM sodium pyruvate, 0.1 mM β-mercaptoethanol, 2.5 g/L glucose.30

### Micro-Column Fabrication

Agarose or agarose-gelatin hydrogels micro-columns were constructed using a three-phase process similar to methods previously described. Briefly, agarose micro-columns were formed using glass capillary tubes (345-701 μm) allowing for the insertion of acupuncture needles (180-350 μm) through the lumen. Molten agarose (3% weight/volume) in Dulbecco's phosphate buffered saline (DPBS) was added to the capillary tube containing the acupuncture needle and allowed the cool. The acupuncture needle was quickly removed to create the hydrogel shell, and the micro-columns were stored in DPBS at 4° C. Agarose-gelatin micro-columns (1.5% agarose+1.5% gelatin) were fabricated as described above except that micro-columns were stored in 7 mL DPBS with 100 μL at room temperature overnight and subsequently washed 3 times in DPBS prior to further experiments. All micro-columns were cut to the appropriate length, UV sterilized for 30 minutes, and stored in DPBS at 4° C.

Micro-columns were transferred to a new petri dish and excess DPBS was removed from the lumen of the micro-column via micropipette and replaced by extracellular matrix (ECM), comprised of 1.0 mg/ml rat tail collagen+1.0 mg/ml mouse laminin (Reagent Proteins, San Diego, CA). DRG explants or motor neuron aggregates were carefully placed at the ends of the micro-columns containing ECM, under stereoscopic magnification using fine forceps and were allowed to adhere for 45 min at 37° C., 5% CO₂. Sensory TE-NMIs were generated by seeding a DRG explant on each end of a micro-column. Motor TE-NMIs were created by seeding a motor neuron aggregate on each end of a micro-column. Mixed motor-sensory TE-NMIs were fabricated by seeding a motor neuron aggregate and a DRG explant on opposite ends of a micro-column. TE-NMIs were then returned to culture and allowed to grow with fresh media replacements every other day.

For mixed TE-NMI characterization, prior to plating, motor neurons were transduced overnight to endogenously express GFP and sensory neurons were transduced overnight to endogenously express tdTomato. For the rest of the in vitro characterization and in vivo implementation, motor neurons were transduced overnight with tdTomato and sensory neurons were transduced overnight with GFP. All TE-NMIs were returned to culture following fabrication with half media changes every other day.

### Immunocytochemistry

TE-NMIs were fixed in 4% paraformaldehyde for 35 minutes, rinsed in 1×PBS, permeabilized with 0.3% Triton X100+4% horse serum in PBS for 60 minutes, and then incubated with primary antibodies overnight at 4° C. Primary antibodies were Tuj-1/beta-III tubulin (T8578, 1:500, Sigma-Aldrich) to label axons and synapsin-1 (A6442, 1:500, Invitrogen) to label pre-synaptic specializations. Following primary antibody incubation, TE-NMIs were rinsed in PBS and incubated with fluorescently-tagged secondary antibodies (1:500; Invitrogen) for 2 h at 18°-24° C. Finally, Hoechst (33342, 1:10,000, ThermoFisher) was added for 10 min at 18°-24° C. before rinsing in PBS. TE-NMIs were imaged on a Nikon A1RSI Laser Scanning confocal microscope paired with NIS Elements AR 4.50.00. Sequential slices of 10-20 μm in the z-plane were acquired for each fluorescent channel. All confocal images presented are maximum intensity projections of the confocal z-slices

### In Vitro TE-NMI Imaging

Phase-contrast microscopy images of TE-NMI were taken over several days in vitro (DIV) to measure neurite length and calculate growth rates. TE-NMI viability and presence of the desired neuronal phenotype(s) were quantified at 10× magnification using a Nikon Eclipse Ti-S microscope, paired with a QIClick camera and NIS Elements BR 4.13.00.

Confocal imaging of TE-NMIs were taken on a Nikon A1RSI Laser Scanning confocal microscope paired with NIS Elements AR 4.50.00. Sequential slices of 10-20 μm in the z-plane were acquired for each fluorescent channel. All confocal images presented are maximum intensity projections of the confocal z-slices.

### Chronic Rodent Sciatic Nerve Axotomy and Acute TE-NMI Transplantation

The capability of TE-NMIs to integrate with the denervated distal nerve was evaluated in a rodent chronic axotomy model. Sprague-Dawley rats were anesthetized with isoflurane and the hind leg cleaned with betadine. Meloxicam (2 mg/kg) was administered subcutaneously in the scruff of the neck and bupivacaine (2 mg/kg) was administered subcutaneously along the incision. The gluteal muscle was separated to expose the sciatic nerve exiting the sciatic notch.

TE-NMIs (3 mm long) were transplanted in the distal nerve using three different surgical paradigms. In a proof-of-concept experiment, an intraneural TE-NMI transplantation was performed in a subset of animals to demonstrate TE-NMI survival following micro-injection. Briefly, the sciatic nerve was exposed as described above. The TE-NMI was loaded into a Hamilton syringe and deposited into the nerve. The epineurium of the sciatic nerve was carefully incised and the needle containing the TE-NMI was inserted into the exposed fascicle, advanced 7 mm into the nerve, and the TE-NMI was deposited within the nerve and the epineurium was closed with 8-0 prolene. The nerve was sharply transected, and the proximal stump was inserted in a nearby muscle. TE-NMI survival was assessed at 2 weeks post transplantation using tissue clearing and multi-photon microscopy.

To evaluate sensory TE-NMI outgrowth and Schwann cell coverage within the otherwise denervated distal nerve, a 5 mm segment of the sciatic nerve was excised, 5 mm proximal to the trifurcatrion, and the proximal nerve was capped with Teflon tape or secured to a nearby muscle. Sensory TE-NMI were placed in a 5 mm nerve wrap (Stryker Orthopedics, Kalamazoo MI) secured to the nerve to provide a protective environment for the nerve and TE-NMI. Approximately 100 μl of 2 mg/ml collagen ECM was applied within the wrap to facilitate outgrowth of the TE-NMI axons in the distal nerve. Animals were randomly assigned to the following groups: (A) one sensory TE-NMI (n=5); (B) two sensory TE-NMIs (n=5), (C) acellular control micro-columns containing ECM only (n=5).

To assess chronic integration with the otherwise denervated muscle, mixed motor-sensory TE-NMI were transplanted in a nerve wrap as described above. In this experiment, the common peroneal nerve was dissected to its proximal origin and the nerve was transected 5 mm distal to the bifurcation. The proximal common peroneal nerve stump was inserted in a nearby muscle. Mixed motor-sensory TE-NMI were placed in a 5 mm nerve wrap (Stryker Orthopedics, Kalamazoo MI) secured to the nerve to provide a protective environment for the nerve and TE-NMI. Approximately 100 μl of 2 mg/ml collagen ECM was applied within the wrap to facilitate outgrowth of the TE-NMI axons in the distal nerve. Animals were randomly assigned to the following groups: (A) TE-NMI (n=5); (B) micro-column control (n=5), (C) no implant control (n=5).

In all procedures, the surgical site was closed with 4-0 absorbable vicryl sutures and skin staples. Animals were recovered and returned to the vivarium for the duration of the study.

### Delayed Axon Fusion

At 20 weeks post transplantation and chronic axotomy, animals were anesthetized and the surgical site was re-exposed. The surgical site was irrigated with calcium free PlasmaLyte-A with a calcium chelating agent, the transplant was isolated and the distal common peroneal nerve was sharply transected. As the transected nerve was bathed with additional PlasmaLyte-A, the tibial nerve was sharpy transected and a standard end-to-end nerve repair was completed using 2 8-0 prolene sutures, securing the proximal tibial nerve with the distal common peroneal nerve. Immediately before tightening the sutures, hypotonic 1% methylene blue solution was applied to the nerve ends, followed by administration of high molecular weight polyethylene glycol (3350 MW). Calcium-containing lactated ringer's solution was applied to the wound to wash away excess PEG. Electrophysiological recordings were performed immediately before and after repair to evaluate acute functional recovery as described below. The deep layers and skin were closed, and the area was dressed as described above.

### Immunohistochemistry

At the terminal time point, animals were euthanized with an intracardial injection of Euthasol. Nerves were extracted and post-fixed in formalin for 24 hours at 4° C., and then rinsed in PBS for another 24 hours. Muscles were extracted in paraformaldehyde for 24 hours at 4° C. and then cryoprotected in 20% sucrose.

For histological assessment following transplantation, the tissue was placed in 30% sucrose overnight, embedded in optimal cutting media, and then frozen in dry ice/isopentane. The transplant site was sectioned longitudinally and a region 5 mm distal to the transplant was sectioned axially at a thickness of 20 μm, mounted on glass slides for staining. Frozen sections were washed three times in PBS, blocked and permeabilized in 4% normal horse serum with 0.3% Triton X-100 for one hour. All subsequent steps were performed using blocking solution for antibody dilutions. Neurons were labeled with chicken anti-MAP2 (1:500, Abcam, ab532) and Schwann cells were labeled with anti-S100 (1:500, Invitrogen, PA1-38585). Primary antibodies were applied overnight at 4° C. followed by the appropriate fluorophore-conjugated secondary antibody (1:1000; AlexaFluor, Invitrogen) for two hours at room temperature. Hoechst was applied (1:10,000) prior to mounting with Fluoromount G and cover slipping. Sensory neurons/axons were visualized by the endogenous GFP expression and motor neurons by the endogenous tdTomato expression.

For cross-sectional histological assessment of the distal nerve following delayed nerve repair, a 1 cm segment of nerve distal to the repair zone was embedded in paraffin. The block was then mounted on a microtome and sectioned axially at a thickness of 8 μm, mounted on glass slides, and prepared for staining as follows. Axial cross-sections were deparaffinized in xylene and rehydrated with a descending gradient of ethanol. Following rehydration, antigen retrieval was performed in TRIS/EDTA buffer for 8 minutes using a modified pressure cooker/microwave technique. Next, normal horse serum in Optimax (Biogenex) was applied to the sections (VectaStain Universal kit per manufacturer's instructions). Sections were incubated overnight at 4° C. with mouse anti-SMI35 (1:1000, Covance, SMI-35R), rabbit anti-S100 (1:500, Invitrogen, PA1-38585), and chicken anti-myelin basic protein (Encor, CPCA-MBP; 1:1500) in Optimax+normal horse serum (VectaStain Universal kit per manufacturer's instructions). After washing the sections three times for 5 minutes with PBS/TWEEN, the appropriate fluorophore-conjugated secondary antibody (1:1000; AlexaFluor, Invitrogen) was applied for one hour at room temperature. After rinsing three times for 5 minutes with PBS/TWEEN, was applied for 20 minutes. Finally, sections were washed as above and cover slipped.

For muscle cross-sectional histological analyses, the tibialis anterior muscle was harvested and stored in 2% paraformaldehyde overnight. Muscles were cryoprotected in 20% sucrose overnight, blocked, frozen, sectioned axially at a thickness of 20 μm, and stained following the protocol described above. To identify muscle actin, sections were incubated with AlexaFluor488-conjugated phalloidin (1:400, Invitrogen, A12379) for two hours at room temperature. Adjacent sections were incubated with rabbit-anti-synaptophysin to identify presynaptic vesicles (1:500, abcam, ab32127) at 4° C. overnight, followed by concurrent application for two hours at room temperature of AlexaFluor-568 antibody (1:500, ThermoFisher, A10042) and AlexaFluor-647-conjugated bungarotoxin to identify postsynaptic receptors (1:1000, Invitrogen, B35450).

### Tissue Clearing

A subset of nerves were extracted for tissue clearing using the Visikol protocol. Briefly, following fixation in formalin for 24 hours at 4° C., nerves were rinsed overnight with PBS at 4° C., dehydrated in a series of ethanol washes for 2 hours each (30%, 50%, 70%, and 90%) and 100% ethanol for 24 hours. Next, nerves were incubated in Visikol 1 for 24 hours followed by Visikol 2 for at least 24 hours to complete the clearing process. TE-NMI survival within the graft region was visualized using multiphoton microscopy (Nikon).

### Functional Assessment

At 16 weeks post axotomy, compound muscle action potential (CMAP) was assessed to evaluate the evoked muscle response. Animals were re-anesthetized and a bipolar subdermal stimulating electrode was placed superficial to the common peroneal nerve. A monopolar subdermal recording electrode was placed in the tibialis anterior and the reference electrode placed in its tendon. The nerve was stimulated (biphasic; amplitude: 0-10 mA; duration: 0.2 ms; frequency: 1 Hz) using a handheld bipolar hook electrode (Rochester Electro-Medical, Lutz, FL; #400900). The supramaximal CMAP recording was obtained and averaged over a train of 5 pulses (100× gain; 10-10,000 Hz band pass and 60 Hz notch filters; Natus Viking EDX). At 20 weeks post axotomy, animals were re-anesthetized and the surgical site was exposed. CMAPs were recorded by stimulating the distal nerve pre delayed nerve repair. Proximal and distal CMAPs were recorded following delayed nerve repair by stimulating 5 mm proximal or distal to the repair site, respectively. Mean peak-to-baseline amplitude were recorded.

To assess the immediate electrical conduction across the repair site, compound nerve action potentials (CNAP) were recorded by stimulating the proximal stump with a bipolar hook electrode and recording with a bipolar hook electrode (1000× gain; 10-10,000 Hz band pass and 60 Hz notch filters; Natus Viking EDX). Mean peak-to-peak amplitude was recorded and conduction velocity was calculated by dividing the distance between the electrodes by the latency.

At 1 month post delayed nerve repair (total 24 weeks following chronic host axotomy), CNAPs and CMAP recordings were obtained as described above.

### Data Acquisition and Statistical Analyses

Neuronal constructs were imaged using phase-contrast or epifluorescence microscopy on a Nikon Eclipse Ti-S with digital image acquisition using a QiClick camera interfaced with Nikon Elements Basic Research software (4.10.01). Fluorescent images were obtained with a Nikon AIR confocal microscope (1024×1024 pixels) with a 10× air objective and 60× oil objective using Nikon NIS-Elements AR 3.1.0 (Nikon Instruments, Tokyo, Japan). Multiple confocal z-stacks were digitally captured and analyzed, with all reconstructions tiled across the full section and full z-stack thickness.

For all TE-NMI neurite outgrowth assays, the longest neurite was measured from the edge of the aggregate (n≥4-6 TE-NMIs per condition per time point). For TE-NMI fabrication characterization, mean neurite outgrowth was compared via a repeated two-way analysis of variance (ANOVA) with cell type and biomaterial hydrogel encasement as the two independent variables at 1 and 3 DIV.

All histological assessments were performed at the graft site (longitudinal frozen), 5 mm distal to the graft site (axial frozen), or 5 mm distal to the delayed nerve repair (axial paraffin). For frozen tissue, TE-NMI neurons/axons were identified as SMI35 negative and GFP positive for sensory neurons/axons or tdTomato positive for motor neurons/axons. For paraffin tissue, SMI35 labeled only the host regenerating/fused axons.

For all axial nerve morphometry quantification, measurements were calculated from confocal z-stack maximum projections and analyzed using FIJI software.46 Automated image processing macros were used to minimize any potential bias. Individual channels were isolated using MaxEntropy thresholding and subsequently quantified using the “Analyze Particles” function on features with an area greater than 1 μm2 to minimize noisy signal. Total count of segmented particles, size of segmented particles, and the percent area covered was calculated from 2-3 sections per animal. Mean values were obtained by averaging the values per animal across groups for further statistical analyses.

For TE-NMI outgrowth and host Schwann cell (S100) reactivity at 6 weeks post transplantation/host axotomy, mean values were compared by one-way analysis of variance (ANOVA) between the following groups: (a) one TE-NMI, (b) two TE-NMI, (c) micro-column only. For the evoked muscle response at 16 weeks post transplantation/host axotomy, mean CMAP amplitude were compared by one-way ANOVA between the following groups: (a) TE-NMI, (b) micro-column only, and (c) injury only/no transplantation.

To quantify the total number of acetylcholine receptors (AchR) and percentage of mature neuromuscular junctions, a researcher blinded to the experimental groups first imaged each muscle section at low magnification to identify regions of bungarotoxin (BGX) positive clusters of AchR (10× air objective, 1024×1024). Next, three regions of interest (ROI) were randomly selected and automatically acquired (2×2 region, 60× oil objective with a 2× digital zoom, 2048×2048) without the researcher visualizing the synaptophysin channel prior to acquisition. Positive BGX cells or the total number of AchR receptors were quantified from the low magnification image from each animal. Mature neuromuscular junctions (NMJs) were identified as BGX positive cells co-labeled with synaptophysin. Mean percent mature NMJs was calculated by dividing the number of mature NMJs by the total number of bungarotoxin positive receptors, averaged across replicates and by group.

For all statistical analyses following delayed nerve repair, since no differences were detected between the negative control groups (micro-column only and injury only/no transplantation), these samples were analyzed as a single group (acellular). Electrical conduction and evoked muscle response immediately after delayed repair (20 weeks following initial host axotomy) and at 1 month post delayed nerve repair (24 weeks following initial host axotomy) were performed by comparing the mean CMAP amplitude, CNAP amplitude, and CNAP velocity using two-tailed unpaired Student's t-tests. Mean host axon count and size, AchR count, and percent mature NMJ were compared at 1 month post delayed nerve repair (24 weeks following initial host axotomy) using two-tailed unpaired Student's t-tests.

When differences existed between groups following one-way ANOVA, post-hoc Tukey's pair-wise comparisons were performed. For all statistical tests, p<0.05 was required for significance and was performed in GraphPad Prism 9 (La Jolla California USA) for Windows 64 bit. Mean values presented as mean±SEM unless otherwise noted.

### Results

### Tissue Engineered Neuromuscular Interfaces (TE-NMIs) are Preformed Axon-Rich Microtissue Within a Protective Biomaterial

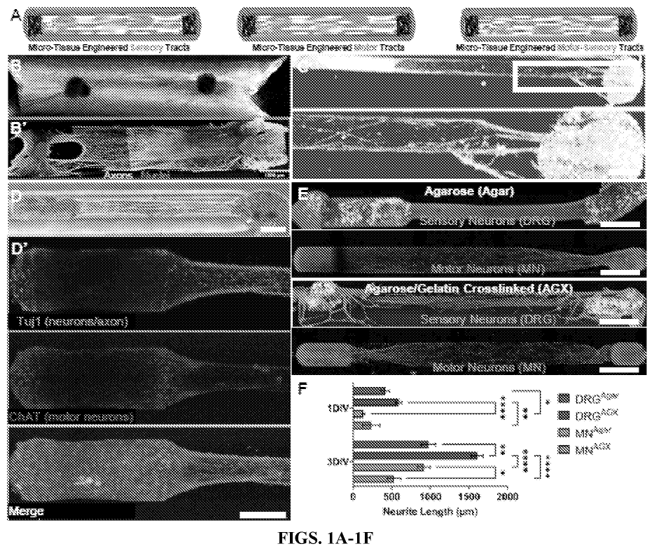

TE-NMIs are anatomically-inspired neural constructs comprised of discrete populations of neurons spanned by long axon tracts similar to the neuronal-axonal organization of the nervous system (FIG. 1). We have engineered microtissue comprised of sensory neurons-axons alone (sensory TE-NMI), motor neurons-axons alone (motor TE-NMI) or a mixed population of motor and sensory neurons-axons (mixed TE-NMI) (FIG. 1A). In an initial experiment, robust sensory axon growth was observed spanning dorsal root ganglia (DRG) spaced 5 mm apart in large macro-scale agarose columns with a 2 mm outer diameter and 1 mm inner diameter containing collagen extracellular matrix (ECM) (FIG. 1B). Next, microtissue engineering fabrication techniques were adapted from methodology previously developed in our lab for Parkinson's disease, spinal cord injury, and brain-machine interfacing. The smallest TE-NMI was 3 mm long with a 350 μm outer and 180 μm inner diameter (FIG. 1C); however, since our goal was to match the fascicular structure in the nerve, unless otherwise noted, TE-NMI in the following studies were 3-5 mm long with a 701 μm outer and 300 μm inner diameter.

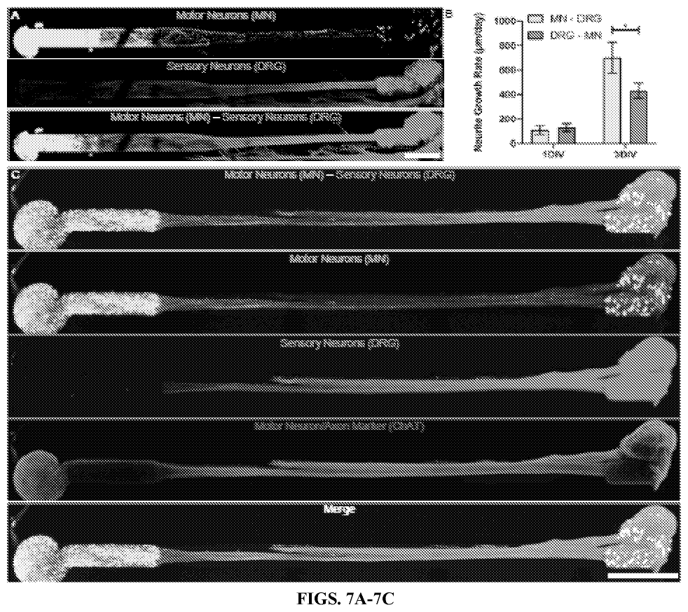

For motor and mixed TE-NMIs, aggregated embryonic spinal motor populations were formed as described previously, and then plated on the end of the micro-column. Healthy neurons and neurite growth were observed via phase-microscopy. TE-NMI immunocytochemistry confirmed the motor neuron phenotype with the co-labeling of Tuj1, a neuronal/axonal marker, and ChAT (FIG. 1D). Agarose is a relatively inert biomaterial but it has a long degradation time into non-resorbable byproducts that may hinder translation. Therefore, alternative bioencasement consisting of an agarose-gelatin composite hydrogel were assessed (FIG. 1E). At 1 day in vitro (DIV), sensory outgrowth in an agarose-gelatin micro-column was faster than motor outgrowth in either an agarose or agarose-gelatin micro-column. However, by 3 DIV, although sensory outgrowth in an agarose-gelatin micro-column was greater than the other groups, increased motor outgrowth was found in the agarose micro-column compared to the agarose-gelatin micro-column (FIG. 1F). These findings corroborate previous work showing sensory axons extending from DRG explants are often faster than motor neurons. Interestingly, these data suggest motor neurons preferentially grow in agarose micro-columns despite the presence of active moieties in the agarose-gelatin microcolumns. To evaluate preferential outgrowth from motor or sensory neurons, neurite outgrowth was measured within a mixed TE-NMI. Faster motor axon growth extending towards the sensory neurons was found at 3 DIV (FIGS. 7A-7C). Based on these findings, agarose micro-columns were selected for in vivo experiments to improve the probability for successful motor outgrowth and provide greater protection from the host immune response following transplantation due the slow degradation rate.

### TE-NMIs Preserve Schwann Cells in an Otherwise Denervated Nerve at 6 Weeks Post Transplantation

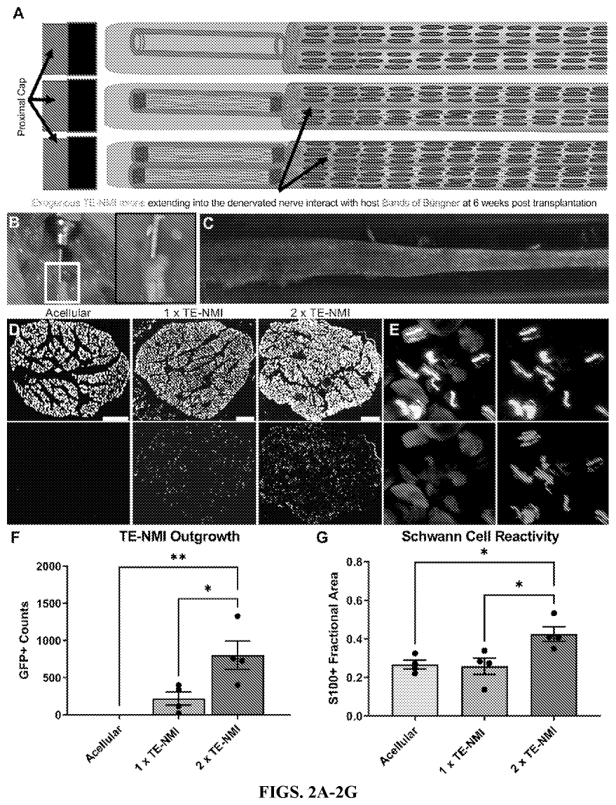

To evaluate whether TE-NMIs can preserve the regenerative capacity of the distal nerve, the sciatic nerve was cut, TE-NMIs were attached to the distal nerve, and the proximal stump was capped to prevent host regeneration (FIG. 2A). In a proof-of-concept experiment, a TE-NMI was micro-injected into the denervated distal nerve by “laying out” the construct (FIG. 2B). At two weeks, robust transplanted TE-NMI neurons and axons were found within the lumen protected by the outer encasement following optical clearing and two-photon microscopy (FIG. 2C).

To test whether TE-NMIs preserve Schwann cell expression, a model of chronic nerve axotomy was used (FIG. 2A). In this study, one or two TE-NMIs were transplanted in a conduit secured the distal sciatic stump. We hypothesized that early reinnervation of the otherwise denervated Schwann cells with TE-NMI axons would preserve Schwann cell expression following prolonged host axotomy. Indeed, at 6 weeks post transplantation, robust sensory TE-NMI axon outgrowth was found extending at least 5 mm within the host tissue (FIG. 2D). Notably, host S100+ Schwann cells closely aligned with GFP+ axons and in some cases, GFP+ axons were visualized extending through Schwann cells resembling bands of Büngner (FIG. 2E). Following implantation of two TE-NMIs, greater GFP+ axonal outgrowth (FIG. 2F) and Schwann cell coverage (FIG. 2G) were observed in the otherwise denervated nerve, whereas decreased Schwann cell expression was observed in the other groups. These findings suggest that exogenous axons extending from TE-NMIs may maintain the pro-regenerative environment necessary for regenerating host axons to reinnervate distal end targets following repair.

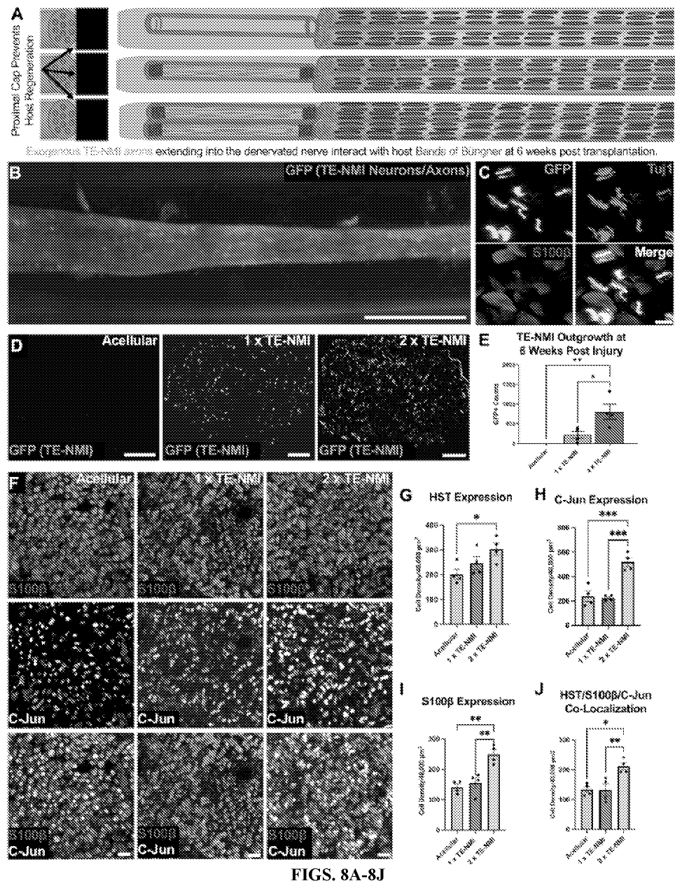

S100β+ Schwann cells and C-Jun expression, in context with total cell counts based on nuclear (Hoechst+) staining, were also quantified at 6 weeks following transplantation using automatic segmentation (FIG. 8F). The total number of Hoechst+ cells was greater in the 2×TE-NMI cohort (303.3±49.35 cells/40,000 μm2) than the acellular group (202.2±41.36 cells/40,000 μm2; F(2, 9)=4.44; p=0.0376; FIG. 8G). Additionally, of the Hoechst+ cells, greater co-expression of S100β was found in the 2×TE-NMI group (249.1±30.29 cells/40,000 μm2) than the 1×TE-NMI (155.2±17.91 cells/40,000 μm2; p=0.0038) or acellular cohorts (141.2±10.22 cells; F(2, 9)=15.78; p=0.0015; FIG. 8I). Similarly, of the Hoechst− and S100β+ cells, greater co-localization with C-Jun was observed in the 2×TE-NMI cohort (249.1±15.14 cells/40,000 μm2) than the 1×TE-NMI (155.2±35.82 cells/40,000 μm2; p=0.0095) or acellular groups (141.2±20.44 cells/40,000 μm2; F(2, 9)=9.883; p=0.0102; FIG. 2J). Interestingly, we also found increased C-Jun expression in the 2×TE-NMI cohort (517.5±71.06 cells/40,000 μm2) compared to the 1×TE-NMI (225.1±23.09 cells/40,000 μm2; p=0.0003) and acellular groups (239.1±83.77 cells/40,000 μm2; F(2, 9)=25.91; p=0.0005; FIG. 8H). These findings suggest that exogenous axons extending from TE-NMIs may maintain the pro-regenerative environment necessary for regenerating host axons to reinnervate distal end targets following repair.

### TE-NMIs Maintain Distal Muscle Target Electrophysiological Activity at 16 Weeks Post Transplantation

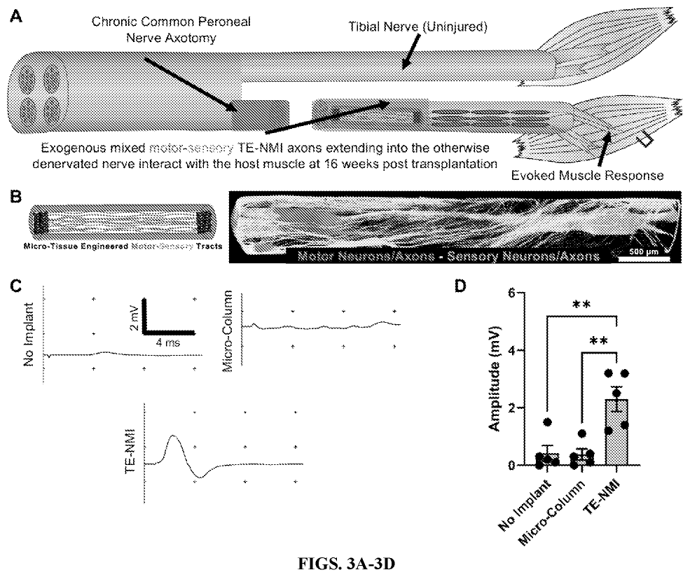

Next, we investigated whether mixed sensory-motor TE-NMIs can preserve the otherwise denervated muscle (FIGS. 3A-3F). The distal common peroneal nerve was selected for the surgical paradigm as its monofascicular nerve architecture more closely matched the TE-NMI size, avoiding the need for multiple constructs. After securing the TE-NMI to the otherwise denervated distal common peroneal nerve, the transected proximal nerve stump was attached to the neighboring muscle to prevent host regeneration (FIG. 3A). Mixed motor-sensory TE-NMIs (FIG. 3B) were chosen as sensory axons were efficacious in preserving Schwann cells, and we postulated that the addition of motor axons will be beneficial to preserve the muscle electrophysiological response following chronic host axotomy. However, based on our previous data, motor neurons co-cultured with sensory neurons appear to grow better in vitro (FIGS. 7A-7C) while improving nerve regeneration, increasing muscle reinnervation, and enabling greater functional recovery.

To test whether mixed TE-NMIs integrated with the otherwise denervated distal muscle target, transcutaneous stimulation was performed at 16 weeks post transplantation. Greater evoked muscle response was observed in the mixed TE-NMI group compared to no implant or micro-column only controls (FIGS. 3C, 3D). These findings indicate that despite the lack of host axons due to the proximal nerve cap preventing host regeneration, TE-NMIs containing motor and sensory neurons project axons to functionally integrate with the otherwise denervated muscle, allowing for a greater electrophysiological muscle response.

### TE-NMIs Provide Exogenous Axons in the Otherwise Denervated Distal Nerve Sheath to Enable Delayed Axon Fusion

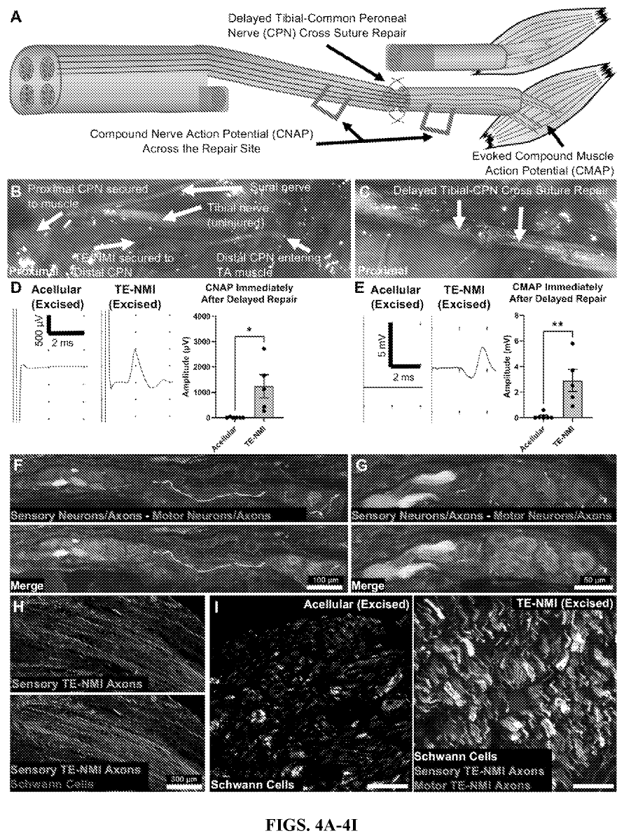

Based on these findings, we hypothesized that TE-NMI axons extending within the otherwise denervated nerve that subsequently integrated with the muscle would be compatible for axon fusion following the standard PEG fusion protocol. At 20 weeks following TE-NMI transplantation, the distal nerve was freshly axotomized for nerve fusion by excising the TE-NMI (FIG. 4B). To test whether TE-NMI transplantation enabled delayed fusion and promoted functional recovery, a cross-suture repair model was utilized to avoid the need for grafting between the contracted proximal and distal stumps and minimizing confounds associated with the prolonged proximal neuron injury. The delayed cross-suture repair was completed by securing the proximal stump of the previously uninjured tibial nerve to the distal end of the freshly axotomized common peroneal nerve containing TE-NMI axons (FIGS. 4A, 4C).

Immediate electrical conduction was obtained following delayed nerve fusion in all TE-NMI animals compared to none of the controls (FIG. 4D). Similarly, greater evoked muscle responses were recorded in the TE-NMI cohort following proximal stimulation (FIG. 4E). Within the harvested transplant site, robust TE-NMI neuron survival was visualized within the micro-column at 20 weeks post repair (FIG. 4F, 4G). Immediately distal to the excised transplant, no host axons were visualized, but sensory and motor axons transduced prior to transplantation were observed extending from the TE-NMI into the host distal nerve (FIG. 4H, 4I).

### TE-NMIs Enable Greater Electrophysiological Recovery, Axon Maturation, and Muscle Reinnervation Following Delayed Nerve Repair

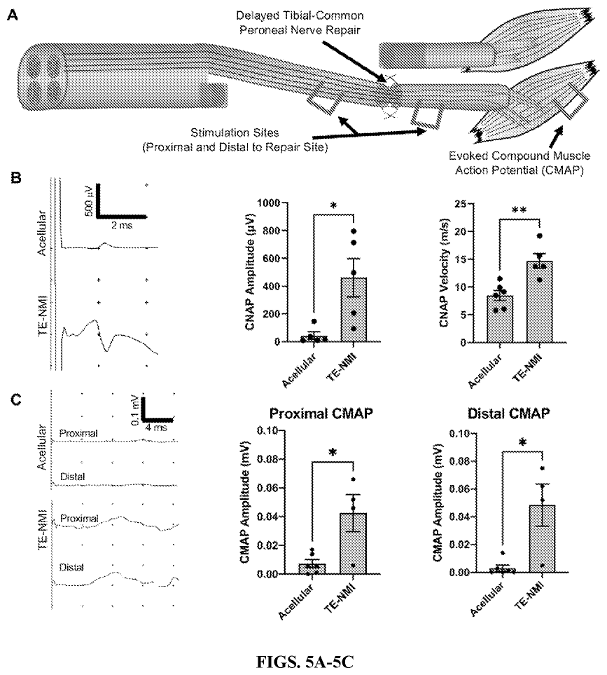

At 1 month post delayed nerve fusion (i.e. 24 weeks after initial nerve transection), greater nerve and muscle electrophysiological functional recovery was found in the TE-NMI group (FIGS. 5B, 5C). Faster conduction velocity was also found in animals that had previously received a TE-NMI prior to delayed nerve repair compared to the acellular controls. In addition, the evoked muscle response was elevated in the TE-NMI group compared to the acellular group. These electrophysiological data indicate at 1 month post delayed nerve repair, the TE-NMI group had greater functional recovery, including nerve conductivity and muscle reinnervation compared to the acellular controls.

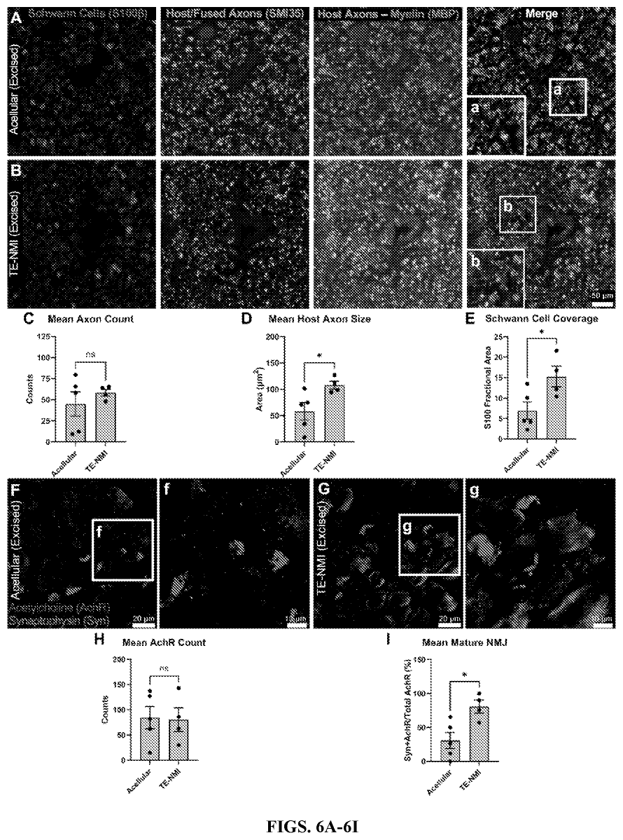

To assess regeneration at 4 weeks post repair, cross-sectional nerve morphometric analyses was completed to identify Schwann cells, host/fused axons, and myelin (FIGS. 6A, 6B). Although there were no differences in the number of host axons distal to the repair site (FIG. 6C), larger host axons were found in the TE-NMI group (FIG. 6D). Greater Schwann cell expression was also found in the TE-NMI group (FIG. 6E). At 1 month post delayed nerve fusion, muscle cross-sections were stained for acetylcholine receptors (bungarotoxin) to identify the neuromuscular junctions (NMJs) and synaptophysin, a presynaptic marker (FIGS. 6F, 6G). Although no significant difference in the total number of acetyl choline receptors (AchR) were found in the target muscle (FIG. 6H), a greater percentage of mature NMJs co-labeling AcHR and synaptophysin were observed following TE-NMI transplantation (FIG. 6I). Further, elevated muscle weight was found in the TE-NMI group compared to the controls (data not shown). Collectively, these findings corroborate the histological data and suggest that TE-NMIs accelerate the ongoing regeneration, maturation, and reinnervation following delayed nerve repair.

In this study, TE-NMIs were developed as a novel implantable microtissue featuring preformed neural networks comprised of discrete populations of motor and sensory neurons spanned by bundled axonal tracts. Following implantation into transected rat nerve, we found that TE-NMI neurons extended numerous axons deep within the host tissue that closely interacted with the endogenous bands of Büngner and resulted in a greater Schwann cell response compared to controls. In addition, we show TE-NMI implants promote functional recovery following delayed nerve repair by preserving the pro-regenerative environment in the distal nerve. Collectively, we report TE-NMIs as the first engineered microtissue designed to prevent the harmful effects of prolonged denervation by providing a source of local axons to innervate the otherwise denervated muscle.

Although slow axon regeneration (1-2 mm/day) is often described as the main challenge for successful functional recovery after nerve injury, two additional important and often underappreciated factors are (1) the capacity for Schwann cells to support the injured proximal neurons and facilitate axon re-growth and (2) the receptiveness of the distal muscle for reinnervation. A prolonged period of time without axon contact in the distal nerve and muscle is a common clinical occurrence, and often occurs in cases of delayed nerve repair, repair of proximal nerve injuries, and/or repair of long-gap nerve injuries. In these cases, prolonged denervation results in the loss of the pro-regenerative environment and target muscle receptiveness necessary for successful regeneration and reinnervation.

To date, there are no commercially available strategies designed to “babysit” or preserve the regenerative capacity of the distal nerve. Innovative surgical techniques have been proposed, such as supercharged end-to-side (SETS) nerve transfers that reroute axons into the denervated distal nerve either closer to the end target or distal to the primary repair. Indeed, SETS may improve functional recovery in challenging nerve repairs by enabling early reinnervation with axons far afield (more distal) to the primary repair site. However, nerve transfers are only indicated in specific scenarios, require transection of an otherwise healthy nerve, and increase the risk for painful neuroma formation. Therefore, TE-NMIs may be more desirable as a more broadly applicable tissue engineering-based approach to “babysitting” that preserves the regenerative capacity of the Schwann cells in the distal nerve as well as target muscle without deliberately transecting an otherwise uninjured nerve.

TE-NMIs are the first preformed microtissue designed to improve functional recovery following nerve repair. These efforts build on previous studies that have shown ectopic neurons transplanted in the distal nerve may preserve the regenerative capacity of the Schwann cells and muscle, and promote functional recovery following delayed nerve repair. In addition, previous work by our group has shown that tissue engineered nerve grafts (TENGs), a stretch-grown living scaffold comprised of neurons and long axonal tracts, also extend neurite processes into the otherwise denervated nerve. TENGs simultaneously facilitate axon regeneration across challenging defects while preserving the regenerative capacity within the distal nerve. While this dual mechanism remains promising for bridging repairs, transplantation requires nerve transection, which for distal nerve babysitting would require disruption of the otherwise intact distal nerve architecture. Therefore, TE-NMIs were developed as a next-generation babysitting strategy that is amenable for minimally invasive delivery.

Nerve fusion has been well described by Bittner and others as a novel approach to immediately restore axon membrane continuity and electrical conduction across coaptation site(s) following repair. These studies also report nerve fusion prevents Wallerian degeneration, minimizes muscle atrophy, and promotes reinnervation, which collectively results in rapid behavioral recovery. While the prospect of nerve fusion remains exciting. it is currently limited to acute nerve injury due to the inevitable Wallerian degeneration, resulting in distal axon degradation that prohibits fusion. In this study, we show the first example of delayed nerve fusion using exogenous TE-NMI axons in the otherwise denervated distal sheath. Successful fusion was achieved by sacrificing the TE-NMI immersed in hypotonic saline, allowing for immediate reconnection between the freshly axotomized distal exogenous axons and the proximal stump. Although immediate electrical conduction was obtained, it is unclear whether axons remain fused at chronic time following fusion. Indeed, at 1 month post fusion, although TE-NMI group had higher functional recovery compared to the acellular controls, there was a stark decrease compared to the initial recovery. It is possible that fused axons broke or pruned during the maturation process. While this study aimed to demonstrate feasibility, future investigation should include longer time points to assess for functional recovery following delayed nerve repair. Here we have shown fusion with exogenous TE-NMI axons in the otherwise denervated distal stump enabled greater functional recovery, nerve maturation, and muscle reinnervation after delayed nerve repair; however, additional studies are necessary to elucidate whether these effects are solely due to delayed nerve fusion or delayed nerve repair. Future work will look towards translation with additional efficacy testing using a human-compatible cell source in a large animal model.

Another important concept to address is that this work demonstrates that tissue engineered neural constructs can integrate with denervated muscle. Innervation plays an important role in development and has been shown to be crucial during the biofabrication process of tissue engineered end-organ or muscle scaffolds. Moreover, future work may include using TE-NMIs as an adjunctive strategy to augment the tissue biofabrication or for other regenerative strategies requiring exogenous axons, such as volumetric muscle loss. Greater functional recovery may be obtainable with additional optimization, such as supplementing TE-NMIs with preformed aligned Schwann cells may enhance motor neuron survival.

Based on these findings, TE-NMIs may represent a transformative approach for restorative peripheral nerve surgery that allows for exogenous axons to provide early muscle reinnervation for enhancing the likelihood for successful recovery following delayed nerve repair. Moreover, the exogenous axons may be spliced in with the host nerve, thus enabling delayed nerve fusion. Collectively, TE-NMIs potentially could offer surgeons an opportunity to improve functional recovery and restore hope for patients with injuries not currently amenable for nerve transfer.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A tissue engineered neuromuscular interface comprising:
an extracellular matrix core; the extracellular matrix core comprising:
a population of neurons at a first end of the extracellular matrix core, the population of neurons having axons extending at least a portion of the way along the extracellular matrix core;
wherein the population of neurons is selected from the group consisting of one or more motor neurons, one or more motor neurons co-cultured with one or more sensory neurons, and a co-aggregate comprising one or more motor neurons and one or more sensory neurons.

2. The tissue engineered neuromuscular interface according to claim 1, further comprising:
a second population of neurons at a second end of the extracellular matrix core, the second population of neurons having axons extending at least a portion of the way along the extracellular matrix core;
the second population of neurons selected from the group consisting of one or more motor neurons, one or more motor neurons co-cultured with one or more sensory neurons, and a co-aggregate comprising one or more motor neurons and one or more sensory neurons.

3. The tissue engineered neuromuscular interface of claim 1, wherein the extracellular matrix core has a largest cross-sectional dimension selected from the group consisting of: between about 10 μm and about 25 μm, between about 25 μm and about 50 μm, between about 50 μm and about 100 μm, between about 100 μm and about 150 μm, between about 150 μm and about 200 μm, between about 200 μm and about 250 μm, between about 250 μm and about 300 μm, between about 300 μm and about 400 μm, between about 400 μm and about 500 μm, between about 500 μm and about 700 μm, and between about 700 μm and about 1000 μm, between about 1000 μm and about 1500 μm, and between about 1500 μm and about 2000 μm, and between about 2000 μm and about 2500 μm, and between about 2500 μm and about 3000 μm.

4. The tissue engineered neuromuscular interface of claim 1, wherein the hydrogel sheath has a largest cross-sectional dimension selected from the group consisting of: between about 20 μm and about 50 μm, between about 50 μm and about 100 μm, between about 100 μm and about 200 μm, between about 200 μm and about 250 μm, between about 250 μm and about 300 μm, between about 300 μm and about 350 μm, between about 350 μm and about 400 μm, between about 400 μm and about 450 μm, between about 450 μm and about 500 μm, between about 500 μm and about 600 μm, between about 600 μm and about 800 μm, between about 800 μm and about 1200 μm, between about 1200 μm and about 1700 μm, and between about 1700 μm and about 2200 μm, and between about 2200 μm and about 2700 μm, and between about 2700 μm and about 3200 μm.

5. The tissue engineered neuromuscular interface of claim 1, wherein the hydrogel sheath has a largest cross-sectional dimension of about 701 μm and the extracellular matrix core has a largest cross-sectional dimension of about 300 μm.

6. The tissue engineered neuromuscular interface of claim 1, wherein the tissue engineered neuromuscular interface has a length between about 100 μm and about 200 μm, between about 200 μm and about 250 μm, between about 250 μm and about 300 μm, between about 300 μm and about 350 μm, between about 350 μm and about 400 μm, between about 400 μm and about 450 μm, between about 450 μm and about 500 μm, between about 500 μm and about 600 μm, between about 600 μm and about 800 μm, between about 800 μm and about 1200 μm, between about 1200 μm and about 1500 μm, and between about 1500 μm and about 2000 μm.

7. The tissue engineered neuromuscular interface of claim 1, further comprising:
one or more non-neuronal cells selected from the group consisting of: endothelial cells, myocytes, myoblasts, astrocytes, olfactory ensheathing cells, oligodendrocytes, or Schwann cells.

8. The tissue engineered neuromuscular interface of claim 1, wherein the neurons are derived from stem cells or are isolated from dorsal root ganglia.

9. The tissue engineered neuromuscular interface of claim 1, wherein the neurons are xenogeneic neurons, autologous/patient-specific neurons, allogenic neurons, whole dorsal root ganglia or sensory explants.

10. The tissue engineered neuromuscular interface of claim 1, wherein the neurons are xenogeneic neurons derived from wild type or transgenic pigs.

11. The tissue engineered neuromuscular interface of claim 1, wherein the extracellular matrix core comprises collagen, gelatin, laminin, fibrin, fibronectin and/or hyaluronic acid.

12. The tissue engineered neuromuscular interface of claim 1, wherein the hydrogel sheath comprises agarose, collagen, gelatin, silk, chitosan, fibrin, and/or hyaluronic acid.

13. A method of preserving the regenerative capacity of a distal nerve segment subsequent to a peripheral nerve injury in a subject in need thereof, the method comprising implanting one or more tissue engineered neuromuscular interface (TE-NMI) into a distal site in the distal nerve segment;
wherein the TE-NMI comprises:
an extracellular matrix core; the extracellular matrix core comprising:
a population of neurons at a first end of the extracellular matrix core, the population of neurons having axons extending at least a portion of the way along the extracellular matrix core;
wherein the population of neurons is selected from the group consisting of one or more motor neurons, one or more motor neurons co-cultured with one or more sensory neurons, and a co-aggregate comprising one or more motor neurons and one or more sensory neurons.

14. The method according to claim 13, wherein the TE-NMI further comprises:
a second population of neurons at a second end of the extracellular matrix core, the second population of neurons having axons extending at least a portion of the way along the extracellular matrix core;
the second population of neurons selected from the group consisting of one or more motor neurons, one or more motor neurons co-cultured with one or more sensory neurons, and a co-aggregate comprising one or more motor neurons and one or more sensory neurons.

15. The method according to claim 13, wherein the implantation is performed immediately after the injury.

16. The method according to claim 15, wherein the injury results from surgery.

17. The method according to claim 13, wherein the implantation is performed less than 24 hours after the injury.

18. The method according to claim 13, wherein the implantation is performed less than 7 days after the injury.

19. The method according to claim 13, wherein the implantation is performed less than 2 weeks after the injury.

20. The method according to claim 13, wherein the implantation is performed less than one month after the injury.

21. The method according to claim 13, wherein the implantation is performed one month or more after the injury.

22. The method according to claim 13, wherein the one or more TE-NMIs are implanted into the distal nerve segment end-to-side, are implanted intrafascicularly, are implanted in-continuity, or in the denervated muscle.

23. The method according to claim 13, wherein implantation of the one or more TE-NMIs is ultrasound- or MRI-guided.

24. The method according to claim 13, wherein at least two tissue engineered neuromuscular interfaces are implanted into the distal nerve segment.

25. The method according to claim 13, wherein at least five tissue engineered neuromuscular interfaces are implanted into the distal nerve segment.

26. The method according to claim 13, wherein at least ten tissue engineered neuromuscular interfaces are implanted into the distal nerve segment.

27. The method according to claim 13, further comprising performing a primary nerve repair procedure to treat the peripheral nerve injury.

28. The method according to claim 27, wherein the primary nerve repair procedure comprises direct anastomosis, autograft, allograft, nerve conduit, nerve transfer, or a tissue engineered nerve graft.

29. A method of treating a peripheral nerve injury in a subject in need thereof, the method comprising:
implanting one or more tissue engineered neuromuscular interface (TE-NMI) into a distal site in the distal nerve segment; wherein the TE-NMI comprises:
an extracellular matrix core; the extracellular matrix core comprising:
a population of neurons at a first end of the extracellular matrix core, the population of neurons having axons extending at least a portion of the way along the extracellular matrix core;
wherein the population of neurons is selected from the group consisting of one or more motor neurons, one or more motor neurons co-cultured with one or more sensory neurons, and a co-aggregate comprising one or more motor neurons and one or more sensory neurons;
monitoring exogenous axonal growth throughout the otherwise denervated distal segment for innervation of muscle and/or sensory end organ;
removing the one or more tissue engineered neuromuscular interface in the distal nerve segment; and
performing a primary nerve repair procedure, thereby treating the peripheral nerve injury.

30. The method according to claim 29, wherein the TE-NMI further comprises:
a second population of neurons at a second end of the extracellular matrix core, the second population of neurons having axons extending at least a portion of the way along the extracellular matrix core;
the second population of neurons selected from the group consisting of one or more motor neurons, one or more motor neurons co-cultured with one or more sensory neurons, and a co-aggregate comprising one or more motor neurons and one or more sensory neurons.

31. The method according to claim 29, wherein the primary nerve procedure comprises direct anastomosis, autograft, allograft, nerve conduit, nerve transfer, or implantation of tissue engineered nerve graft.

32. The method according to claim 29, wherein the TE-NMI is removed less than one week after implantation.

33. The method according to claim 29, wherein the TE-NMI is removed less than one month after implantation.

34. The method according to claim 29, wherein the TE-NMI is removed less than one year after implantation.

35. The method according to claim 29, wherein the TE-NMI is removed one year or more after implantation.

36. A method of treating a peripheral nerve injury in a subject in need thereof, the method comprising:
implanting one or more tissue engineered neuromuscular interface (TE-NMI) into a distal site in the distal nerve segment; wherein the TE-NMI comprises:
an extracellular matrix core; the extracellular matrix core comprising:
a population of neurons at a first end of the extracellular matrix core, the population of neurons having axons extending at least a portion of the way along the extracellular matrix core;
wherein the population of neurons is selected from the group consisting of one or more motor neurons, one or more motor neurons co-cultured with one or more sensory neurons, and a co-aggregate comprising one or more motor neurons and one or more sensory neurons;
monitoring exogenous axonal growth throughout the otherwise denervated distal segment for innervation of muscle and/or sensory end organ;
removing the one or more tissue engineered neuromuscular interface in the distal nerve segment; and
fusing the TE-NMI axons in the distal nerve segment with at least one proximal axon.

37. The method according to claim 36, wherein the TE-NMI further comprises:
a second population of neurons at a second end of the extracellular matrix core, the second population of neurons having axons extending at least a portion of the way along the extracellular matrix core;
the second population of neurons selected from the group consisting of one or more motor neurons, one or more motor neurons co-cultured with one or more sensory neurons, and a co-aggregate comprising one or more motor neurons and one or more sensory neurons.

38. The method according to claim 36, wherein a reagent is applied before removing the TE-NMI to prevent axonal degeneration.

39. The method according to claim 38, wherein the reagent comprises hypotonic saline or a calcium chelating agent.

40. The method according to claim 39, wherein the reagent is hypotonic saline with a calcium chelating agent.

41. The method according to claim 36, wherein a primary nerve repair is performed.

42. The method according to claim 41, wherein the primary nerve procedure comprises direct anastomosis, autograft, allograft, nerve conduit, nerve transfer, or implantation of a tissue engineered nerve graft.

43. The method according to claim 41, wherein a free radical scavenger is applied prior to the primary nerve repair.

44. The method according to claim 43, wherein the free radical scavenger is methylene blue.

45. The method according to claim 41, wherein a fusogen is applied during the primary nerve repair to promote membrane sealing.

46. The method according to claim 43, wherein the fusogen is polyethelene glycol or chitosan.

47. The method according to claim 45, wherein fusogen application promotes nerve regeneration and functional recovery.

48. The composition according to claim 1, wherein the TE-NMI further comprises a hydrogel sheath coaxially surrounding the extracellular matrix core.

49. The method according to claim 13, wherein the TE-NMI further comprises a hydrogel sheath coaxially surrounding the extracellular matrix core.
